(19) United States
(12) Patent Application Publication
(10) Pub. No.: US 2017/0251902 A1
(43) Pub. Date: Sep. 7, 2017

(54) MEDICAL MANIPULATOR

(71) Applicant: OLYMPUS CORPORATION, Tokyo (JP)

(72) Inventors: Shuya JOGASAKI, Tokyo (JP); Masatoshi IIDA, Tokyo (JP); Hiroshi WAKAI, Tokyo (JP)

(73) Assignee: OLYMPUS CORPORATION, Tokyo (JP)

(21) Appl. No.: 15/601,022

(22) Filed: May 22, 2017

Related U.S. Application Data
(63) Continuation of application No. PCT/JP2014/081890, filed on Dec. 2, 2014.

(51) Int. Cl. A61B 1/00; A61B 17/29; A61B 17/22; A61B 1/005

(52) U.S. Cl. CPC A61B 1/0016; A61B 1/0057; A61B 1/0014; A61B 1/00133; A61B 17/29; A61B 17/22031; A61B 2017/22041; G01L 5/226

(57) ABSTRACT

A medical manipulator is provided with: a drive unit provided with a motor; a removable portion that is removably attached to the drive unit and that is provided with a rotating body to be connected to a rotating shaft of the motor; an elongated insertion portion coupled to the removable portion; a distal-end movable part disposed at a distal end of the insertion portion; a wire connecting the rotating body and the distal-end movable part and transferring tension generated by power of the motor to the distal-end movable part to actuate the distal-end movable part; a pressing member that is formed of an elastic material and that presses a longitudinal intermediate position of the wire in the wire diameter direction; and a sensor that is attached to the pressing member and that detects an elastic deformation amount caused in the pressing member according to the tension of the wire.

### CROSS-REFERENCE TO RELATED APPLICATIONS

This is a continuation of International Application PCT/JP2014/081890, with an international filing date of Dec. 2, 2014, which is hereby incorporated by reference herein in its entirety.

### TECHNICAL FIELD

The present invention relates to a medical manipulator.

### BACKGROUND ART

There is a known medical manipulator that is provided with: an actuator portion having a motor; a connection portion that has a pulley to be connected to a rotating shaft of the motor so as to be removably attached to the actuator portion; and a distal-end movement portion (hereinafter, referred to as distal-end movable part) that operates simultaneously with the pulley via a wire, at a distal end of a connecting shaft extending from the connection portion (for example, see PTL 1). This medical manipulator is provided with a tension detecting unit attached to an intermediate position of the wire to detect the tension of the wire by using a strain sensor provided in the tension detecting unit.

### CITATION LIST

### Patent Literature

{PTL 1} Japanese Unexamined Patent Application, Publication No. 2010-46384

### SUMMARY OF INVENTION

According to one aspect, the present invention provides a medical manipulator including: a drive unit that is provided with a motor; a removable portion that is removably attached to the drive unit and that is provided with a rotating body to be connected to a rotating shaft of the motor; an elongated insertion portion that is coupled to the removable portion; a distal-end movable part that is disposed at a distal end of the insertion portion; a wire that connects the rotating body and the distal-end movable part and that transfers tension generated by power of the motor to the distal-end movable part to actuate the distal-end movable part; a pressing member that is formed of an elastic material and that presses a longitudinal intermediate position of the wire in a wire diameter direction; and a sensor that is attached to the pressing member and that detects an elastic deformation amount caused in the pressing member according to the tension of the wire.

### BRIEF DESCRIPTION OF DRAWINGS

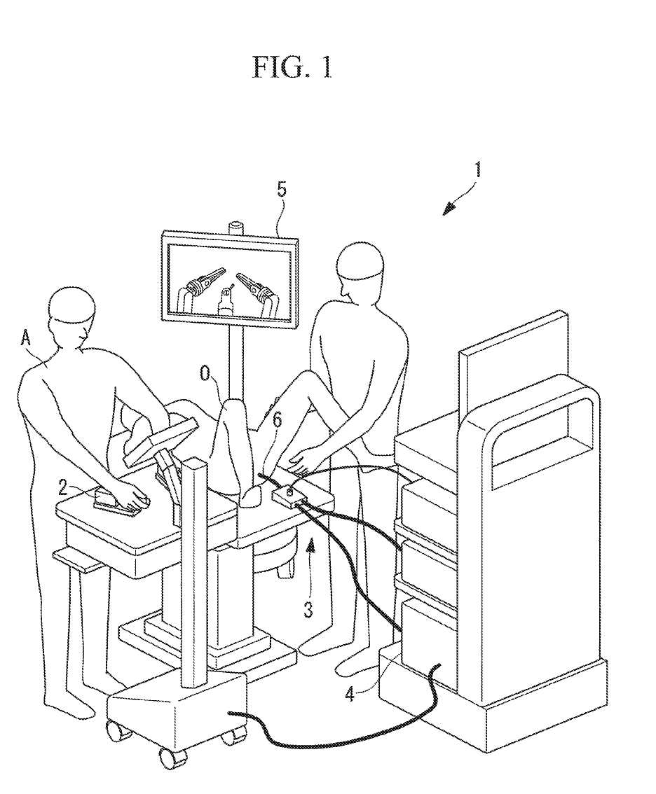

{FIG. 1} FIG. 1 is a view showing the overall configuration of a medical manipulator system according to one embodiment of the present invention.

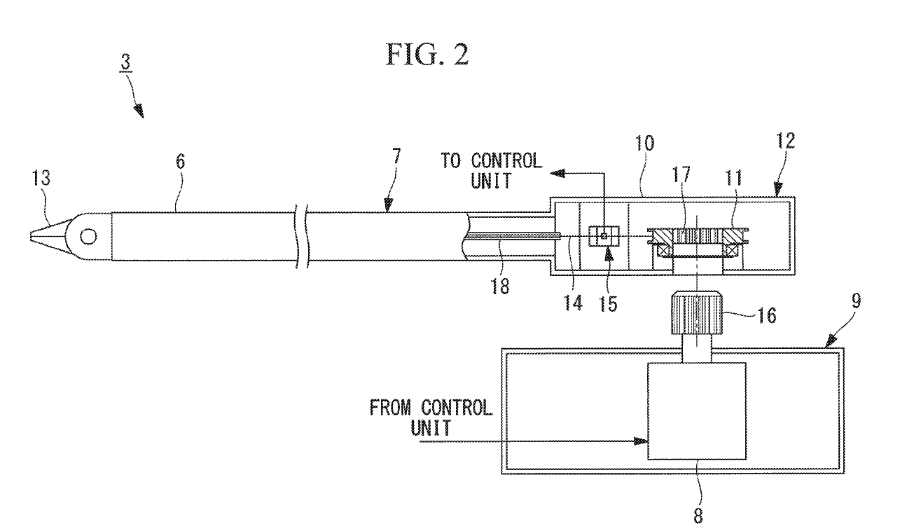

{FIG. 2} FIG. 2 is an exploded view showing, in cutaway fashion, part of a medical manipulator according to the one embodiment of the present invention, provided in the medical manipulator system shown in FIG. 1.

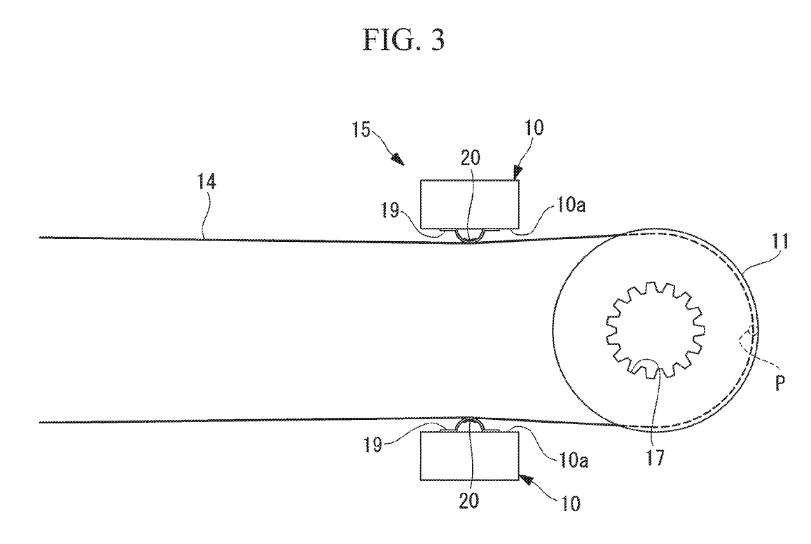

{FIG. 3} FIG. 3 is a plan view showing a wire and a tension detecting unit in the medical manipulator shown in FIG. 2.

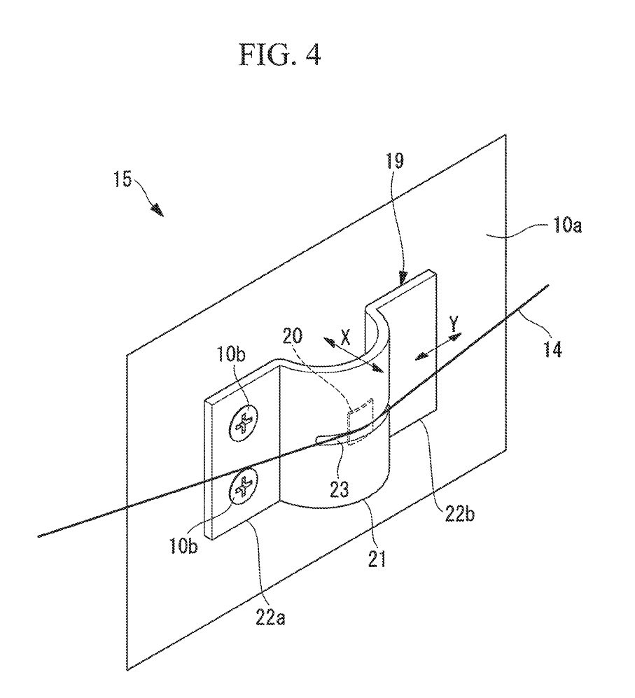

{FIG. 4} FIG. 4 is a perspective view showing the tension detecting unit in the medical manipulator shown in FIG. 2.

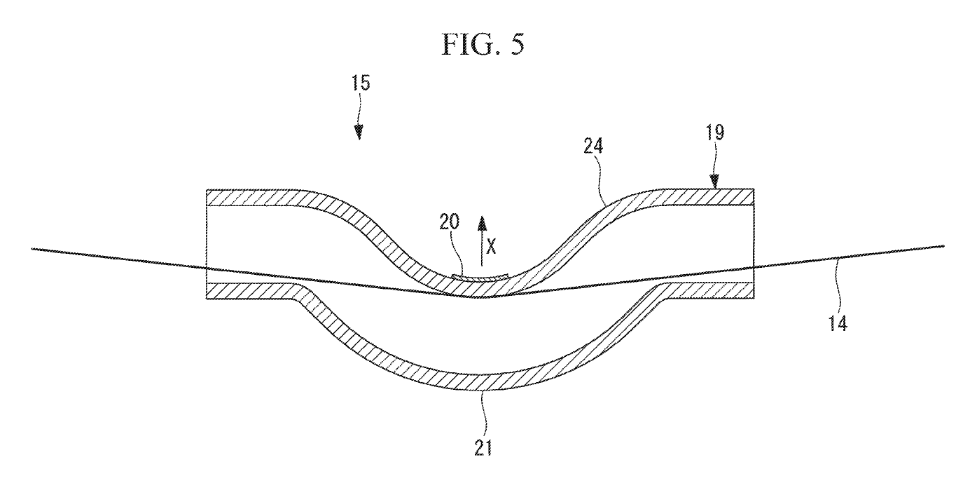

{FIG. 5} FIG. 5 is a longitudinal sectional view showing a modification of the tension detecting unit shown in FIG. 4.

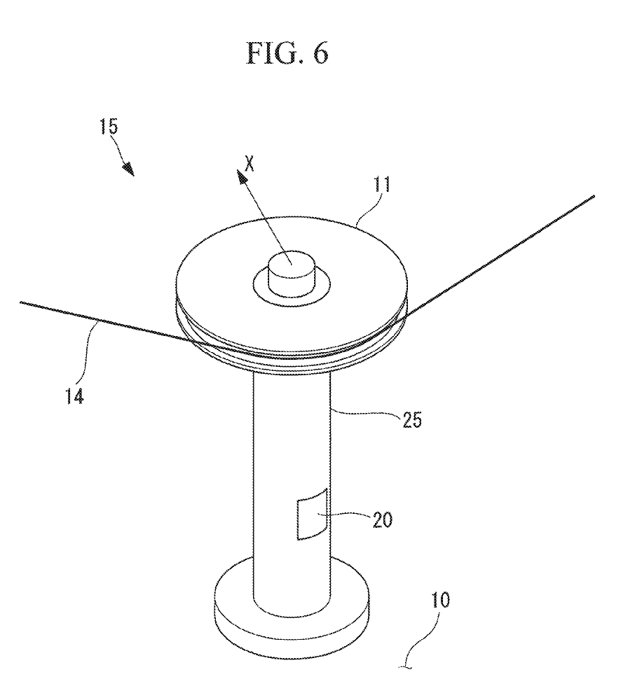

{FIG. 6} FIG. 6 is a perspective view showing another modification of the tension detecting unit shown in FIG. 4.

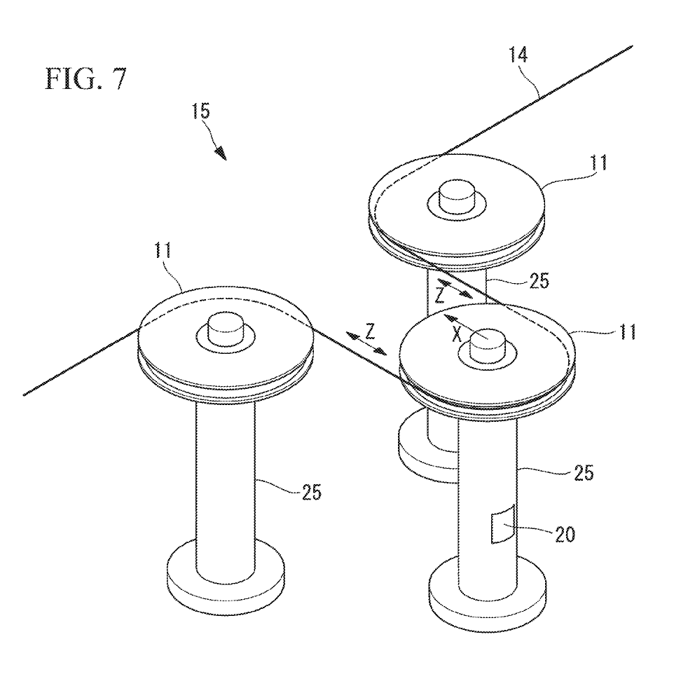

{FIG. 7} FIG. 7 is a perspective view showing still another modification of the tension detecting unit shown in FIG. 4.

### DESCRIPTION OF EMBODIMENT

A medical manipulator 3 according to one embodiment of the present invention will be described below with reference to the drawings.

As shown in FIG. 1, for example, a medical manipulator system 1 of this embodiment is provided with: a master device 2 that is operated by an operator A; the medical manipulator 3 that is inserted into a body cavity of a patient O; a control unit 4 that controls the medical manipulator 3 on the basis of an operation input that is input to the master device 2; and a monitor 5.

As shown in FIG. 2, the medical manipulator 3 of this embodiment is provided with: a manipulator body 7 that has an elongated flexible insertion portion 6 to be inserted into the body cavity; and a drive unit 9 that is removably attached to the manipulator body 7 and that has a motor 8.

The manipulator body 7 is provided with: a removable portion 12 that has a base (base member) 10 fixed to a base end of the insertion portion 6 and a pulley (rotating body) 11 rotatably attached to the base 10; a distal-end movable part 13, such as straight grasping forceps, that is disposed at a distal end of the insertion portion 6; a wire 14 that connects the pulley 11 of the removable portion 12 and the distal-end movable part 13; and a tension detecting unit 15 that detects the tension of the wire 14.

The rotating shaft of the motor 8 is provided with a spline shaft 16. Furthermore, the pulley 11 is provided with a spline hole 17 with which the spline shaft 16 of the motor 8 is removably engaged.

Furthermore, as shown in FIG. 3, the wire 14 is looped around the pulley 11, and the wire 14 is fixed to the pulley 11 at a fixed point P through welding or the like. The wire 14 is inserted into a guide sheath 18 and is made to pass through the insertion portion 6.

The tension detecting unit 15 is provided with: pressing members 19 that are disposed at longitudinal intermediate positions located between the pulley 11 and the distal-end movable part 13; and sensors 20 that are formed of strain gauges placed on the pressing members 19.

As shown in FIG. 4, each of the pressing members 19 is formed of a leaf spring member that is provided with: a curved portion 21 curved in a convex shape in one direction that is formed by bending a strip-shaped member; and flat portions 22a and 22b extending toward both ends of the curved portion 21. One flat portion 22a is fixed to a flat side surface 10a of a mounting member fixed to the base 10, with screws 10b, for example, and the other flat portion 22b is not fixed but is disposed so as to be movable along the side surface 10a.

Furthermore, a groove 23 for accommodating the wire 14 is formed on a convex-side outer surface of the curved portion 21 at the center in the width direction thereof. Furthermore, the sensor 20 is placed on the opposite surface of the curved portion 21 from the groove 23. The sensor 20 detects elastic deformation caused in the curved portion 21 and outputs it to the control unit 4.

Furthermore, each of the pressing members 19 is disposed at a longitudinal intermediate position of the wire 14 extending from the pulley 11 toward the distal-end movable part 13 and presses the wire 14 in the wire diameter direction, as shown in FIG. 3, while accommodating the intermediate position of the wire 14 in the groove 23 of the curved portion 21.

Specifically, when the tension exerted on the wire 14 is increased, the wire 14 presses the curved portion 21, with which the wire 14 is brought into contact, in the direction of arrow X; thus, the curved portion 21 is elastically deformed in the direction in which it collapses, while displacing the flat portion 22b, which is not fixed, in the direction of arrow Y.

The sensor 20 outputs a signal corresponding to the elastic deformation of the pressing member 19, thus detecting the tension caused in the wire 14.

The control unit 4 controls the movement of the medical manipulator 3 on the basis of the tension of the wire 14 detected by the sensor 20. Specifically, when the insertion portion 6 of the medical manipulator 3 is curved, the wire 14 inserted into the insertion portion 6 via the guide sheath 18 is brought into more contact with the inner surface of the guide sheath 18, thus increasing the friction force.

Therefore, because it is necessary to exert a larger tension on the wire 14 in order to activate the distal-end movable part 13 against the friction force, the control unit 4 performs control such that the driving force of the motor 8 with respect to an operation input that is input to the master device 2 is increased when the tension received from the sensor 20 is increased and such that the driving force of the motor 8 is decreased when the tension is decreased.

The operation of the thus-configured medical manipulator 3 of this embodiment will now be described.

In order to perform treatment for an affected area in the body cavity by using the medical manipulator 3 of this embodiment, the operator A inserts the insertion portion 6 of the medical manipulator 3 into the body cavity of the patient O from the distal-end movable part 13 and makes the distal-end movable part 13 face the affected area while observing, on the monitor 5, an image acquired by a separately inserted endoscope.

Next, the operator A operates the master device 2, thereby inputting the operation amount in the master device 2 to the control unit 4, the control unit 4 causes the motor 8 to generate the driving force corresponding to the operation amount, and thus the pulley 11 coupled to the rotating shaft is rotationally driven in one direction. Accordingly, the tension exerted on one portion of the wire 14 is increased, and the tension exerted on the other portion of the wire 14 is decreased, thereby causing the distal-end movable part 13, which is connected to the ends of the wire 14, to perform opening movement or closing movement.

In this case, if tensions that are enough to keep the wire 14 from sagging are exerted thereon, the wire 14 remains in the state in which it is accommodated in the grooves 23, which are formed on the outer surfaces of the curved portions 21 of the pressing members 19, and the tensions can be continuously detected by the sensors 20. Then, an increase in the tensions exerted on the wire 14 causes an increase in the pressing forces of the wire 14 in the directions in which the convex shapes of the curved portions 21 of the leaf spring members, with which the intermediate positions of the wire 14 are brought into contact, collapse, and the elastic deformations of the curved portions 21 caused by the forces are detected by the sensors 20.

The detected elastic deformations are output from the sensors 20 to the control unit 4 and are used in the control unit 4 to control the medical manipulator 3. Specifically, in this embodiment, when the tensions exerted on the wire 14 are increased, because the control unit 4 increases the driving force of the motor 8, the wire 14 is pulled against the friction force, thereby providing an advantage of being able to accurately operate the distal-end movable part 13, which is disposed at the distal end of the insertion portion 6.

Furthermore, according to the medical manipulator 3 of this embodiment, because the tensions of the wire 14 are detected by using deformations of the pressing members 19, which are fixed to the bases 10, there is an advantage that it is not necessary to limit the stroke of the wire 14 and to use movable wiring as the wiring for power supply to the sensors 20 and signal extraction.

Note that, in this embodiment, although each of the pressing members 19 is formed by bending the strip-shaped leaf spring, instead of this, as shown in FIG. 5, it is also possible to bend a pipe 24 through which the wire 14 is made to pass, to press the wire 14 in the wire diameter direction by using the inner surface of the curved portion 21, and to detect, with the sensor 20, a deformation of the pipe 24 caused by the tension of the wire 14. By doing so, even when the tension exerted on the wire 14 is decreased, thereby causing sagging of the wire 14, there is an advantage that it is possible to prevent the wire 14 from falling off from the pipe 24 and to reliably detect the tension of the wire 14 when the tension is next increased.

Furthermore, in this embodiment, the tension detecting unit 15 makes the wire 14 slide on each pressing member 19, which is formed of a leaf spring, thereby detecting the tension of the wire 14 on the basis of the elastic deformation of the pressing member 19. Instead of this, as shown in FIG. 6, as the tension detecting unit 15, it is possible to dispose, at a distal end of a shaft 25 that is fixed to the base 10 in a cantilever manner, a pulley 11 that is supported so as to be capable of rotating about the axis of the shaft 25, to attach a sensor 20 to the shaft 25, and to loop an intermediate position of the wire 14 around the pulley 11.

By doing so, because the pulley 11 is rotated with respect to the shaft 25 when the wire 14 is pulled and moved in the longitudinal direction, the friction exerted on the wire 14 in the tension detecting unit 15 can be reduced. Because the cantilevered shaft 25 is bent due to an elastic deformation thereof when the tension is exerted on the wire 14, the elastic deformation is detected by the sensor 20, thereby making it possible to indirectly detect the tension of the wire 14.

Furthermore, it is also possible to prepare three shafts 25 each having a pulley 11, shown in FIG. 6, to arrange the three shafts 25 in parallel so as not to arrange the axes thereof in the same plane, as shown in FIG. 7, and to loop the wire 14 around the three pulleys 11 on opposite sides thereof in an alternating manner. As shown in FIG. 7, because the wire 14 is bent in a substantially U-shaped manner by the pulley 11 located at the center, the directions of the tension exerted on the wire 14 (arrows Z) can be aligned with the direction of curvature of the shaft 25 (arrow X). Specifically, the sensor 20 is disposed on the shaft 25 that supports the pulley 11 located at the center, thereby providing an advantage that the tension exerted on the wire 14 can be more reliably detected. Note that the sensor 20 may be disposed at the other one of the shafts 25.

Furthermore, it is also possible to dispose a battery in the drive unit 9 side and to dispose contact points at which wires are connected in order to transfer power in the battery to the sensors 20 when the drive unit 9 and the manipulator body 7 are coupled, between the drive unit 9 and the manipulator body 7. Furthermore, a signal to be output from the sensor 20 may be extracted from the manipulator body 7 to the outside via an electrical contact point between the drive unit 9 and the manipulator body 7 or may be extracted wirelessly.

As a result, the above-described embodiment leads the following aspects.

According to one aspect, the present invention provides a medical manipulator including: a drive unit that is provided with a motor; a removable portion that is removably attached to the drive unit and that is provided with a rotating body to be connected to a rotating shaft of the motor; an elongated insertion portion that is coupled to the removable portion; a distal-end movable part that is disposed at a distal end of the insertion portion; a wire that connects the rotating body and the distal-end movable part and that transfers tension generated by power of the motor to the distal-end movable part to actuate the distal-end movable part; a pressing member that is formed of an elastic material and that presses a longitudinal intermediate position of the wire in a wire diameter direction; and a sensor that is attached to the pressing member and that detects an elastic deformation amount caused in the pressing member according to the tension of the wire.

According to this aspect, when the removable portion, which can be removably attached to the drive unit, is attached thereto, the rotating body of the removable portion is connected to the rotating shaft of the motor, the rotating body is rotated due to a driving force of the motor, and the wire, one end of which is connected to the rotating body, is wound by the rotating body, thus causing a tension in the wire. Because the distal-end movable part, which is disposed at the distal end of the elongated insertion portion, is connected to the other end of the wire, when a tension is caused in the wire, the distal-end movable part is activated by the tension.

In this case, because the pressing member, which is provided at a longitudinal intermediate position of the wire, presses the wire in the wire diameter direction, when the tension of the wire is fluctuated, the force of the wire pressing back the pressing member is fluctuated. Specifically, when the tension of the wire is increased, the force of pressing back the pressing member is increased; thus, the pressing member, which is formed of an elastic material, is elastically deformed, and the elastic deformation amount is detected by the sensor. Accordingly, the tension caused in the wire can be indirectly detected by the sensor. In this case, because the sensor is not required to be fixed to the wire, it is possible to accurately control the distal-end movable part by detecting the tension of the wire without limiting the stroke of the wire and without using movable wiring.

In the above-described aspect, the pressing member may be a leaf spring that is elastically deformed according to the tension of the wire.

By doing so, because the wire can be reliably pressed by a spring force of the leaf spring, and the leaf spring is elastically deformed in proportion to the force of the wire pressing back, the tension can be accurately detected by the sensor.

Furthermore, in the above-described aspect, a base member that supports the rotating body in a manner allowing it to rotate may be further provided; the pressing member may be formed of a shaft one end of which is fixed to the base member; and a pulley that is attached to the other end of the shaft so as to be capable of rotating and around which the intermediate position of the wire is looped may be further provided.

By doing so, when a tension is caused in the wire through driving of the motor, thus moving the wire in the longitudinal direction, the pulley, around which the intermediate position of the wire is looped, is rotated, thus making it possible to perform smooth movement without interfering with movement of the wire. Furthermore, when the force of the wire pressing back is imposed on the pulley, because the shaft, to which the pulley is attached at the distal end thereof, is elastically deformed, the elastic deformation amount of the shaft is detected by the sensor, thereby making it possible to indirectly detect the tension of the wire.

Furthermore, in the above-described aspect, it is also possible to further include: a base member that supports the rotating body in a manner allowing it to rotate; three shafts, one ends of which are fixed to the base member and that have parallel axes disposed in a positional relation in which the axes are not arranged in the same plane; and pulleys that are attached to the other ends of the shafts in a manner allowing them to rotate about the axes and around which the wire is looped on opposite sides thereof in an alternating manner, wherein the pressing member is formed of one of the shafts.

By doing so, the wire is looped around the pulleys on the opposite sides thereof in an alternating manner, the pulleys being provided on the ends of the three shafts, which have parallel axes that are not arranged in the same plane, thereby making it possible to bend the wire in a substantially U-shaped manner by using the pulley located at the center. Accordingly, the directions of the tension exerted on the wire are aligned with the direction of elastic deformation of the shaft, thereby making it possible to more reliably detect the tension.

Furthermore, in the above-described aspect, the pressing member may be formed into a bent tube shape through which the wire is made to pass.

By doing so, the tension of the wire made to pass through the tubular pressing member can be detected by using elastic deformation of the pressing member.

According to the present invention, an advantageous effect is afforded in that it is possible to accurately control the distal-end movable part by detecting the tension of the wire without limiting the stroke of the wire and without using movable wiring.

### REFERENCE SIGNS LIST

3 medical manipulator
6 insertion portion
8 motor
9 drive unit
10 base (base member)
11 pulley (rotating body)
12 removable portion
13 distal-end movable part
14 wire
19 pressing member
20 sensor
25 shaft (pressing member)

1} A medical manipulator comprising:
a drive unit that is provided with a motor;
a removable portion that is removably attached to the drive unit and that is provided with a rotating body to be connected to a rotating shaft of the motor;
an elongated insertion portion that is coupled to the removable portion;
a distal-end movable part that is disposed at a distal end of the insertion portion;
a wire that connects the rotating body and the distal-end movable part and that transfers tension generated by power of the motor to the distal-end movable part to actuate the distal-end movable part;
a pressing member that is formed of an elastic material and that presses a longitudinal intermediate position of the wire in a wire diameter direction; and
a sensor that is attached to the pressing member and that detects an elastic deformation amount caused in the pressing member according to the tension of the wire.

2} A medical manipulator according to claim 1, wherein the pressing member is a leaf spring that is elastically deformed according to the tension of the wire.

3} A medical manipulator according to claim 1,
wherein a base member that supports the rotating body in a manner allowing it to rotate is further provided;
the pressing member is formed of a shaft one end of which is fixed to the base member; and
a pulley that is attached to the other end of the shaft so as to be capable of rotating and around which the intermediate position of the wire is looped is further provided.

4} A medical manipulator according to claim 1, further comprising:
a base member that supports the rotating body in a manner allowing it to rotate;
three shafts, one ends of which are fixed to the base member and that have parallel axes disposed in a positional relation in which the axes are not arranged in the same plane; and
pulleys that are attached to the other ends of the shafts in a manner allowing them to rotate about the axes and around which the wire is looped on opposite sides thereof in an alternating manner,
wherein the pressing member is formed of one of the shafts.

5} A medical manipulator according to claim 1, wherein the pressing member is formed into a bent tube shape through which the wire is made to pass.
